(19) United States
(12) Patent Application Publication
(10) Pub. No.: US 2017/0007228 A1
(43) Pub. Date: Jan. 12, 2017

(54) RETRACTOR AND RETRACTOR BLADE

(71) Applicant: Alphatec Spine, Inc., Carlsbad, CA (US)

(72) Inventor: Jonathan T. Costabile, San Diego, CA (US)

(21) Appl. No.: 15/204,558

(22) Filed: Jul. 7, 2016

Related U.S. Application Data
(60) Provisional application No. 62/189,636, filed on Jul. 7, 2015.

(51) Int. Cl. A61B 17/02

(52) U.S. Cl. CPC A61B 17/0218; A61B 17/0206; A61B 2017/00867

(57) ABSTRACT

A retractor blade and a retractor blade kit are provided. The retractor blade has a proximal end, a distal end, and a toe-out mechanism between the proximal end and the distal end. The toe-out mechanism, which is part of the blade, moves the distal end from an insert position to a toe-out position when activated. The retractor blade kit has a first blade with a first length and a second blade with a second length that is less than the first length. The first blade and the second blade have about the same stiffness such that each blade deflects about the same amount when a distal end of each blade is subjected to the same force.

### CROSS-REFERENCE TO RELATED APPLICATIONS

This application claims priority to U.S. Provisional Patent Application No. 62/189,636 filed on Jul. 7, 2015, which is incorporated in its entirety herein by reference.

### FIELD OF TECHNOLOGY

The instant application is related to retractors used in minimally invasive surgical procedures, and in particular, retractor blades that counter or mitigate deflection caused by musculature loading.

### BACKGROUND

Retractors with at least two blades are used to provide a surgical corridor during minimally invasive surgical (MIS) procedures. The blades typically have a proximal end secured to a retractor frame and a distal end that is inserted into and positioned within a surgical site of a patient. The distal ends within the surgical site are subjected to forces from surrounding muscle, skin, etc., commonly referred to as musculature load. The musculature load deflects or flexes the distal ends of the blades in an inward direction which in turn narrows the surgical corridor. Adjustment mechanisms with springs, rotation devices, and the like have been used to counter the flexing of the blades caused by the musculature load. However, such adjustment mechanisms increase the complexity of the retractor and its operation during surgery. Therefore, a retractor with blades that offset or mitigate musculature load during a surgical procedure without the use of springs, rotation devices, and the like would be desirable.

### SUMMARY

A retractor blade with a proximal end, a distal end and a toe-out mechanism integrally formed with the retractor blade and positioned between the proximal end and the distal end is provided. The toe-out mechanism pivots the distal end radially outward from an insert position to a toe-out position when activated. The distal end of the retractor blade may pivot radially outward by an angle between 1-45°, preferably between 5-25°. The toe-out mechanism may be made from a shape-memory alloy (SMA) that has a transformation temperature and pivots the distal end radially outward from the insert position to the toe-out position when a temperature of the SMA is raised from a first temperature to above the transformation temperature to a second temperature. The first temperature may be less than or equal to about ambient room temperature and the second temperature may be greater than or equal to about 35° C. The retractor blade may have a first blade portion and a second blade portion with the first blade portion formed from the SMA and the second blade portion formed from a non-SMA material. In embodiments, the first blade portion extends generally parallel to the second blade portion along the retractor blade. In other embodiments, the first blade portion is co-axial with the second blade portion. The retractor blade may include a third blade portion formed from the non-SMA material with the first blade portion positioned between the second blade portion and the third blade portion such that the first blade portion is positioned between the proximal end and the distal end of the blade.

A retractor blade system with at least two blades having different lengths but generally the same stiffness is also provided. A first blade has a first length and is made from a first material having a first elastic modulus and a second blade has a second length that is less than the first length and is made from the first material and a second material that has a second elastic modulus that is than the first elastic modulus. The first blade and the second blade have the same stiffness such that a distal end of the first blade and a distal end of the second blade deflect an equal amount when an equal and/or predefined force is applied to the distal end of the first blade and the distal end of the second blade. The first blade and the second blade may have a layered structure. The second blade has a layer made from the first material and a layer made from the second material. The first blade may be made from a single layer of the first material. In embodiments, the second blade has a pair of outer layers made from the first material and an inner layer made from the second material.

Additional features and advantages of the retractor blades described herein will be set forth in the detailed description which follows, and in part will be readily apparent to those skilled in the art from that description or recognized by practicing the embodiments described herein, including the detailed description which follows, the claims, as well as the appended drawings.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this application. The drawings illustrate the various embodiments described herein, and together with the description serve to explain the principles and operations of the claimed subject matter.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be better understood when read in conjunction with the following drawings where like structure is indicated with like reference numerals and in which:

FIG. 1A schematically depicts a retractor blade with a first blade portion formed from a shape memory alloy (SMA) and a second blade portion formed from a non-SMA material according to one or more embodiments disclosed and described herein;

FIG. 1B schematically depicts the first blade portion and the second blade portion shown in FIG. 1A attached to each other;

FIG. 1C schematically depicts a front view of the blade shown in FIG. 1B;

FIG. 1D schematically depicts a pair of retractor blades as depicted in FIGS. 1B-1C inserted within a surgical site with a toe-out mechanism activated according to one or more embodiments disclosed and described herein;

FIG. 2A schematically depicts a front view of a retractor blade according to one or more embodiments disclosed and described herein;

FIG. 2B schematically depicts a side view of the retractor blade shown in FIG. 2A;

FIG. 2C schematically depicts a pair of retractor blades as depicted in FIGS. 2A-2B inserted within a surgical site with a toe-out mechanism activated according to one or more embodiments disclosed and described herein;

FIG. 3A schematically depicts a front view of a retractor blade according to one or more embodiments disclosed and described herein;

FIG. 3B schematically depicts a side view of the retractor blade shown in FIG. 3A;

FIG. 3C schematically depicts a pair of retractor blades as depicted in FIGS. 3A-3B inserted within a surgical site with a toe-out mechanism activated according to one or more embodiments disclosed and described herein;

FIG. 4A schematically depicts a front view of a retractor blade according to one or more embodiments disclosed and described herein;

FIG. 4B schematically depicts a side view of the retractor blade shown in FIG. 4A;

FIG. 4C schematically depicts a pair of retractor blades as depicted in FIGS. 4A-4B with a toe-out mechanism activated according to one or more embodiments disclosed and described herein;

FIG. 5A schematically depicts a front view of a retractor blade according to one or more embodiments disclosed and described herein;

FIG. 5B schematically depicts a side view of the retractor blade shown in FIG. 5A;

FIG. 5C schematically depicts a pair of retractor blades as depicted in FIGS. 5A-5B with a toe-out mechanism activated according to one or more embodiments disclosed and described herein;

FIG. 6 graphically depicts transformation temperature as a function of annealing temperature for a Ni—Ti SMA;

FIG. 7 schematically depicts three retractor blades having different lengths and exhibiting constant deflection when an equal and/or predefine force is applied to a distal end of each retractor blade according to one or more embodiments disclosed and described herein;

FIG. 8A schematically depicts a perspective view of a retractor blade according to one or more embodiments disclosed and described herein;

FIG. 8B schematically depicts the cross-section 8B-8B labeled in FIG. 8A;

FIG. 9A schematically depicts a finite element (FE) model showing deflection as a function of resultant force applied to the retractor blade with a length of 180 mm;

FIG. 9B schematically depicts a FE model showing deflection as a function of resultant force applied to an optimized retractor blade with a length of 100 mm;

FIG. 10A schematically depicts a pair of retractor blades according to one or more embodiments disclosed and described herein; and

FIG. 10B schematically depicts a pair of retractor blades according to one or more embodiments disclosed and described herein.

### DETAILED DESCRIPTION

A retractor blade (also referred to simply as “blade” herein) that resists or mitigates musculature load during MIS procedures is provided. The retractor blade has a proximal end, a distal end, and a toe-out mechanism between the proximal end and the distal end. The toe-out mechanism is integral with the blade and pivots the distal end radially outward from an insert position to a toe-out position when activated. The toe-out mechanism may be in the form of a shape memory alloy (SMA) that pivots the distal end radially outward when activated. The blade may include a first blade portion and a second blade portion. The first blade portion may be formed or made from the SMA and the second blade portion may be formed or made from a non-SMA material. In embodiments, the first blade portion may extend generally parallel with the second blade portion along the blade. In other embodiments, the first blade portion may be co-axial with the second blade portion. The first blade portion may be activated by raising a temperature of the SMA to above a transformation temperature of the SMA.

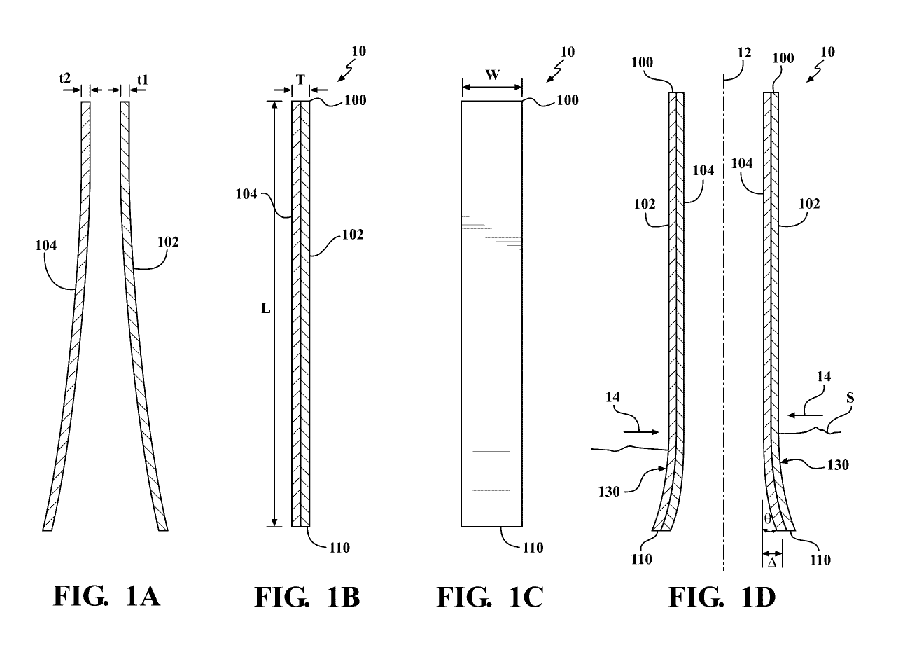

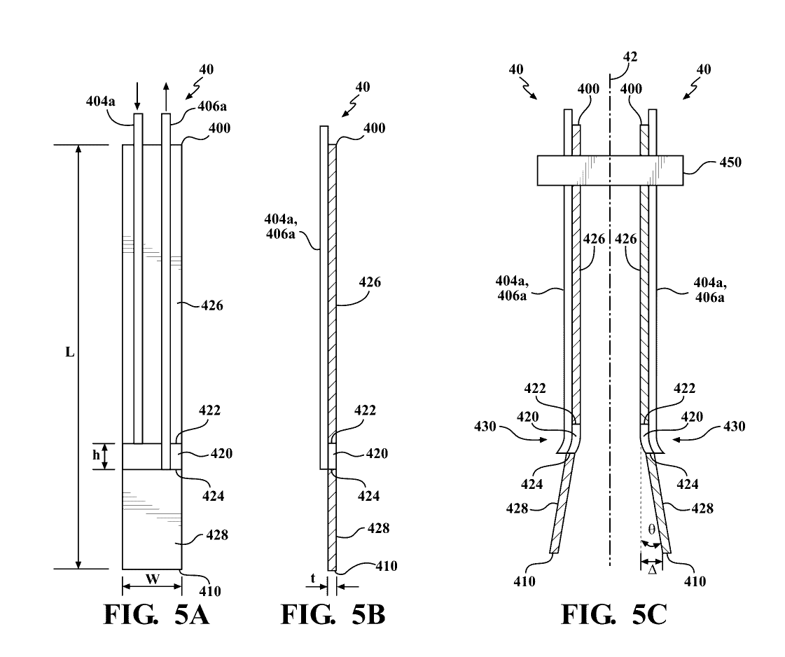

Referring to FIGS. 1A-1D, a side view of a first blade portion 102 and a second blade portion 104 used to make a retractor blade 10 (FIG. 1B) are shown in FIG. 1A; a side view of the retractor blade 10 made from the first blade portion 102 and second blade portion 104 is shown in FIG. 1B; a front view of the retractor blade 10 is shown in FIG. 1C; and a pair of retractor blades 10 within a surgical site are shown in FIG. 1D. The first blade portion 102 has a thickness ‘t₁’ and the second blade portion has a thickness ‘t₂’. The thickness t₁ of the first blade portion 102 may be generally equal to the thickness t₂ of the second blade portion, or in the alternative, the thickness t₁ may not be generally equal to the thickness t₂. The retractor blade 10 is made by attaching the first blade portion 102 to the second blade portion 104 as shown in FIG. 1B. The first blade portion 102 may be attached to the second blade portion 104 using any attachment mechanism or process known in the art, illustratively including attaching the first blade portion 102 to the second blade portion 104 with adhesive(s), mechanical fastener(s), welding, diffusion bonding at the like. Attached together, the first blade portion 102 and the second blade portion 104 form the retractor blade 10. The retractor blade 10 has a total thickness ‘T’ and a length ‘L’. The first blade portion 102 may be made or formed from a SMA, illustratively including a nickel-titanium (Ni—Ti) alloy with generally equal proportions of Ni and Ti in atomic percent. Such Ni—Ti alloys may be commercially known as Nitinol. The second blade portion 104 may be formed or made from a non-SMA material, illustratively including steels, stainless steels, titanium alloys, polymers, carbon fibers, ceramics, composites of polymers with impregnated carbon fibers, etc. When the first blade portion 102 and the second blade portion 104 are not attached to each other as shown in FIG. 1A, each blade portion may have a predetermined shape that is generally nonlinear. However, when the first blade portion 102 is attached to the second blade portion 104, the shape of the retractor blade 10 in the length L direction may be generally linear. The generally linear shape in the length L direction is referred to herein as an “insert position.” The shape of the retractor blade 10 in a width ‘W’ direction (not shown) may be generally linear, or in the alternative, may have an arcuate shape such as an arc shape with an outer convex surface and an inner concave surface. Referring to FIG. 1D, a pair of retractor blades 10 are shown inserted into a surgical site with a distal end 110 of each retractor blade 10 positioned beneath skin ‘S’ of a patient. Proximal ends 100 of the retractor blades 10 are located outside of the surgical site and are typical supported by and attached to a retractor frame (not shown) that firmly holds the proximal ends 100 in a fixed position relative to each other. The portion of the retractor blades 10 positioned within the surgical site and beneath the skin S of the patient have a toe-out mechanism 130 (the SMA located beneath the skin S) that pivots the distal ends 110 of the retractor blades 10 radially outward from a central axis 12 extending between the retractor blades 10. It should be appreciated that the portion of the retractor blades 10 beneath the skin S of the patient are in contact with the patient's muscle, fat, blood vessels, etc. Accordingly, the temperature of the portion of the retractor blades 10 beneath the skin S of the patient will increase from an ambient temperature of an operating room where a surgical procedure is being performed to a temperature near or equal to the body temperature of the patient. For example, ambient temperature for an operating room is typically between 20° C. to 24° C. and normal patient body temperature is between 36.5° C. to 37.5° C. Accordingly, there is generally a minimum 10° C. temperature difference between the ambient operating temperature and a patient's body temperature. When the temperature of the first blade portion 102 of the retractor blades 10 beneath the skin S of the patient reaches a desired transformation temperature between 0 and 40° C., preferably between 20-24° C. and 36.5-37.5° C., the toe-out mechanism 130 (the SMA) of each retractor blade 10 is activated and transforms from a low temperature SMA phase to a high temperature SMA phase. The temperature of the SMA may be set at a desired temperature as shown in FIG. 5. Particularly, FIG. 5 graphically depicts transformation temperature (labeled ‘Af’ in the figure) as a function of annealing temperature for a Ti-50.85 Ni (at %) alloy annealed for 30 minutes followed by air cooling (Moorlehhem, W. V. and Otte, D., “The Use of Shape Memory Alloys for Fire Protection” Duerig, T. W. et al. (Eds.) Engineering Aspects of Shape Memory Alloys, Butterworth-Heinemann Ltd., London, 1990). It should be appreciated that the transformation temperature for the toe-out mechanism 130 formed or made from the SMA may be set at a temperature between 0° C. and 40° C., e.g., between 25° C. and 35° C., by annealing the SMA at an annealing temperature that provides a desired transformation temperature. For example, FIG. 5 illustrates annealing a Ti-50.85 Ni (at %) alloy at about 500° C. for 30 minutes followed by air cooling provides a transformation temperature of about 32° C. for the alloy.

Below the transformation temperature the SMA has or exists in a low temperature SMA phase (martensite for a Ni—Ti SMA) and above the transformation temperature the SMA has or exists in a high temperature SMA phase (austenite for a Ni—Ti SMA). Accordingly, increasing the SMA from a first temperature below the transformation temperature to a second temperature above the transformation temperature results in a transformation of the SMA from the low temperature SMA phase to the high temperature SMA phase, which in turn results in a reversible strain of the material (up to 8% for a Ni—Ti SMA). It should be appreciated that although the discussion herein is in relation to raising a temperature of the SMA to above a transformation temperature, the temperature of the SMA may be lowered from a first temperature above the transformation temperature to a second temperature below the transformation temperature such that the SMA is transformed from the high temperature SMA phase to the low temperature SMA phase, which in turn results in a reversible strain of the material (up to 8% for a Ni—Ti SMA).

The reversible strain of the SMA pivots the distal ends 110 radially outward by an angle ‘θ’ and/or a distance ‘Δ’ relative to a central axis 12 located between the retractor blades 10. The distal end 110 pivoted radially outward by the angle θ and/or the amount (distance) Δ is referred to herein as the ‘toe-out’ position. The angle θ that the distal end 110 pivots in a non-stressed state may be greater than about 1° and less than about 45°, preferably greater than about 5° and less than about 25°. Also, it should be appreciated that the toe-out mechanism 130 may have a higher strength when in the toe-out position than when in the insert position. For example, the low temperature SMA phase for Nitinol (martensite) has a typical yield strength of about 100 MPa and the high temperature SMA phase for Nitinol (austenite) has a typical yield strength of about 560 MPa, i.e. about a five-fold increase in strength. Accordingly, the toe-out mechanism 130 not only pivots the distal end 110 radially outward when above the transformation temperature, also but exhibits at least a five-fold increase in strength to assist in mitigating musculature load inward forces on the retractor blade 10.

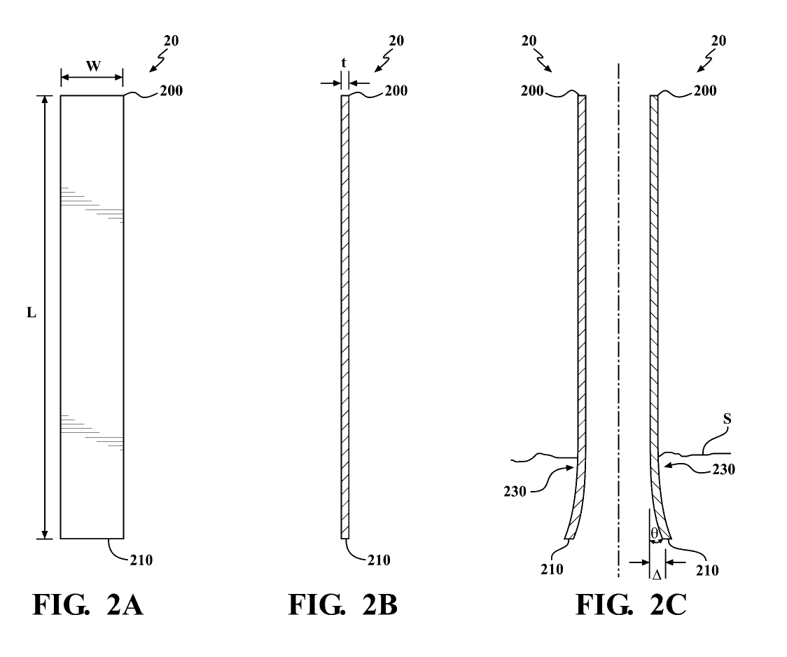

In embodiments, a retractor blade 20 may be made entirely from a SMA as shown in FIGS. 2A-2C. The retractor blade 20 has a proximal end 200, a distal end 210, a thickness t, a width W, and a length L. Extending from the proximal end 200 to the distal end 210 is a blade portion formed or made form a SMA. Referring to FIG. 2C, a pair of retractor blades 20 are shown inserted into a surgical site with the distal end 210 of each retractor blade 20 positioned beneath skin ‘S’ of a patient. Proximal ends 200 of the retractor blades 20 are located outside of the surgical site and are typical supported by and attached to a retractor frame (not shown) that firmly holds the proximal ends 200 in a fixed position relative to each other. The portion of the retractor blades 20 positioned within the surgical site and beneath the skin S of the patient have a toe-out mechanism 230 similar to the toe-out mechanism 230 discussed above that pivots the distal ends 210 of the retractor blades 20 radially outward from a central axis 22 extending between the retractor blades 20. Similar to the retractor blades 10, the temperature of the portion of the retractor blades 20 beneath the skin S of the patient will increase above an ambient temperature of an operating room where a surgical procedure is being performed. When the temperature of the portion of the retractor blades 20 beneath the skin S of the patient reaches a desired transformation temperature between 0-40° C., e.g., between 25° C. and 35° C., the toe-out mechanism 230 (the SMA beneath the skin S) of each retractor blade 20 is activated and transforms from a low temperature SMA phase to a high temperature SMA phase. The transformation from the low temperature SMA phase to the high temperature SMA phase results in a reversible strain in the SMA which pivots the distal ends 210 radially outward from a central axis 22 located between the retractor blades 20 by an angle θ and/or an amount (distance) Δ. The angle θ that the distal end 110 pivots in a non-stressed state may be greater than 1° and less than 45°, preferably greater than about 5° and less than about 25°. Also, it should be appreciated that similar to the toe-out mechanism 130 discussed above, the toe-out mechanism 230 may have higher strength when in the toe-out position than when in the insert position. Accordingly, the toe-out mechanism 230 not only pivots the distal end 210 radially outward when above the transformation temperature, but also but exhibits at least a five-fold increase in strength to assist in mitigating musculature load inward forces on the retractor blade 20.

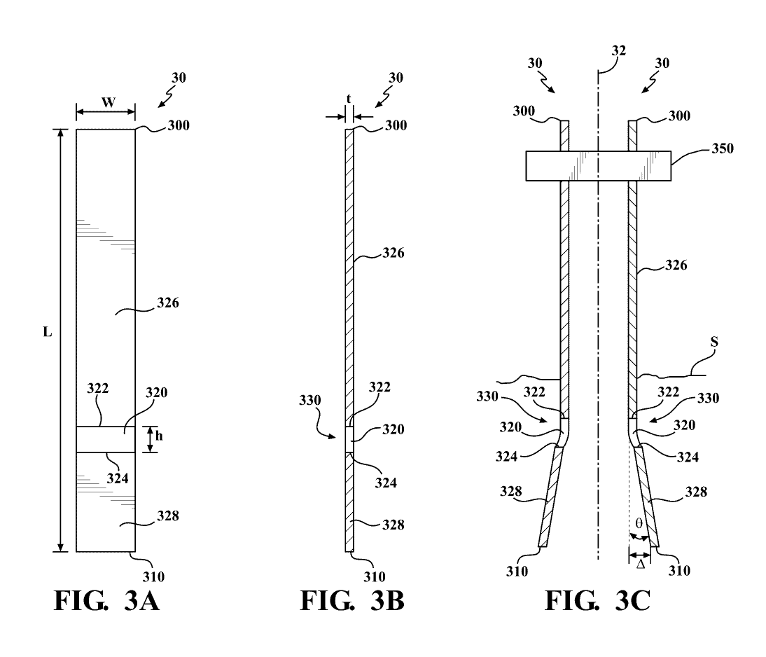

Referring to FIGS. 3A-3C, in embodiments, a retractor blade 30 may have a first blade portion formed or made from an SMA that is coaxial with a second blade portion that is formed or made from a non-SMA material. Particularly, the retractor blade 30 has a proximal end 300, a distal end 310, a thickness t, a width W, and a length L. Located between the proximal end 300 and the distal end 310 is a first blade portion 320 having a length ‘h’ and formed or made from a SMA. The first blade portion has a proximal side edge 322 and a distal side edge 324. Extending from the proximal end 300 to the proximal side edge 322 of the first blade portion 320 is a second blade portion 326 formed or made from a non-SMA material illustratively including steels, stainless steels, titanium alloys, polymers, carbon fibers, ceramics, composites of polymers with impregnated carbon fibers, etc. Extending from the distal end 310 to the distal side edge 324 of the first blade portion 320 is a third blade portion 328 formed or made from a non-SMA material illustratively including steels, stainless steels, titanium alloys, polymers, carbon fibers, ceramics, composites of polymers with impregnated carbon fibers, etc. In embodiments, the second blade portion 326 and the third blade portion 328 are formed or made from the same non-SMA material. In other embodiments, the second blade portion 326 and the third blade portion 328 are not formed or made from the same non-SMA material. Although FIGS. 3A-3C show the first blade portion 320 spaced apart from the distal end 310, it should be appreciated that the first blade portion 320 may extend from the second blade portion 326 completely to the distal end 310. That is, the retractor blade 30 may have a first blade portion 320 with a distal side edge 324 that is also the distal end 310 of the retractor blade 30, and the first blade portion 320 extends from the distal end 310 to the second blade portion 326 with the proximal end 300. The first blade portion 320 may be attached to the second blade portion 326 and/or the third blade portion 328 using any attachment mechanism or process known in the art, illustratively including using adhesive(s), mechanical fastener(s), welding, diffusion bonding at the like. Referring to FIG. 3C, a pair of retractor blades 30 are shown inserted into a surgical site with a distal end 310 and the first blade portion 320 of each retractor blade 30 positioned beneath skin ‘S’ of a patient. Proximal ends 300 of the retractor blades 30 are located outside of the surgical site and are supported by and attached to a retractor frame 350 that firmly holds the proximal ends 300 in a fixed position relative to each other. The first blade portion 320 positioned within the surgical site and beneath the skin S of the patient serves has a toe-out mechanism 330 (the first blade portion 320) that pivots the distal ends 310 of the retractor blades 30 radially outward from a central axis 32 extending between the retractor blades 30. It should be appreciated that the toe-out mechanism 330 beneath the skin S of the patient is in contact with the patient's muscle, fat, blood vessels, etc. Accordingly, the temperature of the toe-out mechanism 330 beneath the skin S of the patient will increase above an ambient temperature of an operating room where a surgical procedure is being performed. For example, the toe-out mechanism 330 will increase in temperature from an ambient temperature for an operating room (20-24° C.) to about or slightly less than a normal patient body temperature (36.5-37.5° C.). When the temperature of the toe-out mechanism 330 of the retractor blades 30 beneath the skin S of the patient reaches a desired transformation temperature between 0-40° C., e.g., between 25° C. and 35° C., the toe-out mechanism 330 of each retractor blade 30 is activated and transforms from a low temperature SMA phase to a high temperature SMA phase. The transformation from the low temperature SMA phase to the high temperature SMA phase results in a reversible strain in the SMA which pivots the distal ends 310 radially outward from a central axis 32 located between the retractor blades 30 by an angle θ and/or an amount (distance) ‘Δ’. The angle θ that the distal end 110 pivots in a non-stressed state may be greater than about 1° and less than about 45°, preferably greater than about 5° and less than about 25°. Also, it should be appreciated that the first blade portion 320 may have a higher strength when in the toe-out position than when in the insert position as discussed above in reference to retractor blades 10 and 20.

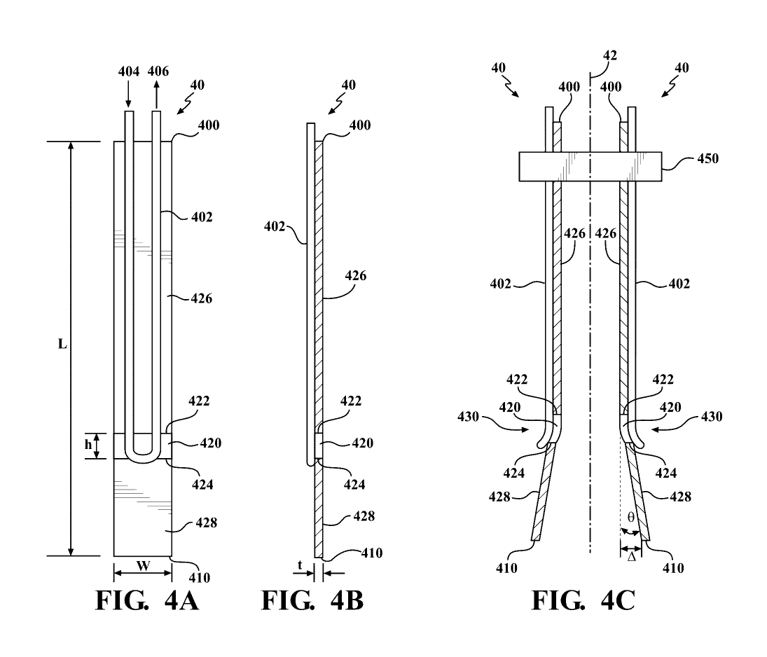

Referring to FIGS. 4A-4C, in embodiments a retractor blade 40 may be activated by an external stimulus, i.e. a stimulus provided instead of or in addition to an increase in temperature caused by a patient's body heat. Particularly, the retractor blade 40 has a proximal end 400, a distal end 410, a thickness t, a width W, and a length L. Located between the proximal end 400 and the distal end 410 is a first blade portion 420 having a length ‘h’ and formed or made from a SMA. The first blade portion 420 has a proximal side edge 422 and distal side edge 424. Extending from the proximal end 400 to the proximal side edge 422 of the first blade portion 420 is a second blade portion 426 formed or made from a non-SMA material illustratively including steels, stainless steels, titanium alloys, polymers, carbon fibers, etc. Extending from the distal end 410 to the distal side edge 424 of the first blade portion 420 is a third blade portion 428 formed or made from a non-SMA material illustratively including steels, stainless steels, titanium alloys, polymers, carbon fibers, ceramics, composites of polymers with impregnated carbon fibers, etc. In embodiments, the second blade portion 426 and the third blade portion 428 are formed or made from the same non-SMA material. In other embodiments, the second blade portion 426 and the third blade portion 428 are not formed or made from the same non-SMA material. The first blade portion 420 may be attached to the second blade portion 426 and/or the third blade portion 428 using any attachment mechanism or process known in the art, illustratively including using adhesive(s), mechanical fastener(s), welding, diffusion bonding at the like. Extending along the length L of the retractor blade 40 and in contact with the first blade portion 420 is a conduit 402. The conduit 402 has an inlet 404 and an outlet 406. Fluid, e.g. water, at a desired temperature may flow into the conduit 402 through inlet 404 and flow out of the conduit 402 through the outlet 406. The fluid may have a temperature greater than the first blade portion 420 and heat may be transferred from the fluid in the conduit 402 to a toe-out mechanism 430 (the first blade portion 420) of the blade 40. In embodiments, the fluid has a temperature such that sufficient heat is transferred from the conduit 402 to the toe-out mechanism 430 such that the temperature of the SMA in the first blade portion 420 (toe-out mechanism 430) increases from a first temperature below the transformation temperature of the SMA to a second temperature above the transformation temperature of the SMA. Increasing the temperature of the SMA from the first temperature to the second temperature activates the toe-out mechanism 430 and the distal ends 410 of the retractor blades 40 are pivoted radially outward from a central axis 42 extending between the retractor blades 40 by an angle θ and/or an amount (distance) Δ (FIG. 4C). In this manner the toe-out mechanism 430 is not required to be beneath the skin S of a patient in order for the toe-out mechanism 430 to be activated. The angle θ that the distal end 110 pivots in a non-stressed state may be greater than about 1° and less than about 45°, preferably greater than about 5° and less than about 25°. The amount Δ for the distal end 310 in a non-stressed state (without musculature loading) may be greater than about 1 mm and less than about 25 mm, preferably greater than about 5 mm and less than about 15 mm. Also, it should be appreciated that the first blade portion 420 may have a higher strength when in the toe-out position than when in the insert position as discussed above in reference to the retractor blades 10, 20 and 30.

Referring to FIGS. 5A-5C, in embodiments the retractor blade 40 may be activated by a different external stimulus that the stimulus described above with respect to FIGS. 4A-4C. Particularly, extending along the length L of the retractor blade 40 and in contact with the first blade portion 420 is a first electrical wire 404a and a second electrical wire 406a. The first electrical wire 404a is in electrical contact with the proximal side edge 422 and the second electrical wire 406a is in electrical contact with the distal side edge 424. An electrical current passes through the first electrical wire 404a, through the first blade portion 420 and through the second electrical wire 406a. Passing the electrical current through the first blade portion 420 results in Joule (resistive) heating of the SMA and increases the temperature of the first blade portion 420 from a first temperature below the transformation temperature of the SMA to a second temperature above the transformation temperature of the SMA, thereby providing the toe-out mechanism 430. Accordingly, the toe-out mechanism 430 is activated and the distal ends 410 of the retractor blades 40 are pivoted radially outward from a central axis 42 extending between the retractor blades 40 by the angle θ and/or the amount (distance) Δ. In this manner the first blade portion 420 is not required to be beneath the skin S of a patient in order for the SMA to increase in temperature and experience the transformation from the low temperature SMA phase to the high temperature SMA phase.

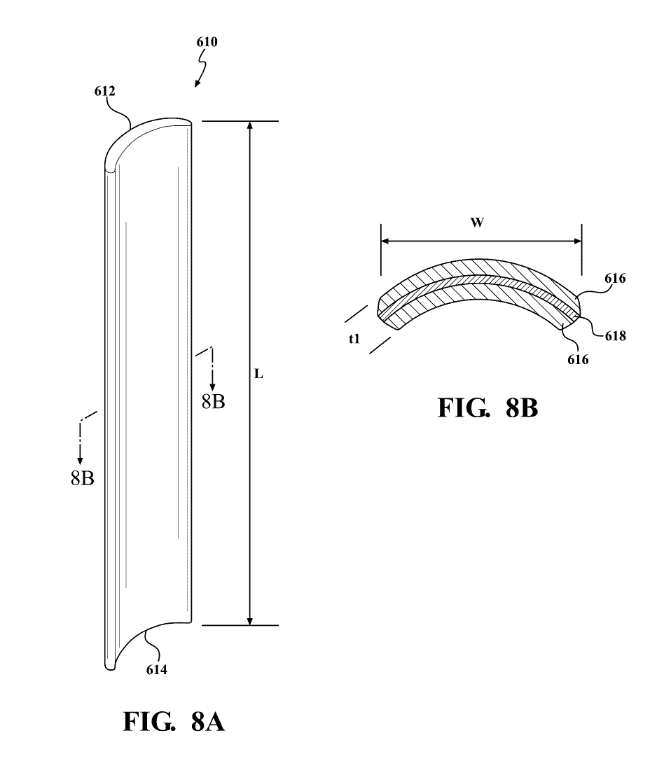

Referring to FIGS. 7-8B, a retractor system or retractor blade kit with retractor blades of different lengths exhibiting a constant amount of deflection when subjected to a given musculature load is shown. As used herein the term “deflection” refers to the angle and/or distance a distal end of a retractor blades moves relative to an axis extending parallel to the retractor blade when the retractor blade is in an un-stressed generally linear state. Particularly, three retractor blades 610, 620, 630 are shown in FIG. 7. The retractor blade 610 has a first length L1, the retractor blade 620 has a second length L2 that is greater than the first length L1, and the retractor blade 630 has a third length L3 that is greater than the second length L2. The widths (not shown) of each retractor blade 610, 620, 630 are approximately equal and the thicknesses (t1) of each retractor blade 610, 620, 630 are approximately equal. The retractor blades 610, 620, 630 have proximal ends 612, 622, 632, respectively, and distal ends 614, 624, 634, respectively. Upon subjecting the distal ends 614, 624, 634 of the retractor blades 610, 620, 630, respectively, to a force of P1, the stiffness of each retractor blade 610, 620, 630 is the same and thus the deflection of each blade (d1) is the same. The stiffness of a given retractor blade may be defined as:

k=AE/L (1)

where k is the stiffness, A is the cross-sectional area (W×t1) of the retractor blade, E is the elastic modulus (tensile) of the retractor blade (also known as Young's modulus) and L is the length of the retractor blade. It should be appreciated from expression (1) that two retractor blades having different lengths but the same stiffness will exhibit the same amount of deflection when a proximal end of each retractor blade is held secure and a given load is applied to an unsecured distal end of each retractor blade. It should also be appreciated that if the two retractor blades with the same stiffness have the same width and thickness (same cross-sectional area A), the elastic modulus E must vary as the lengths of the retractor blades vary. The elastic modulus of a given material is defined as:

E=σ(ε)/ε (2)

where σ(ε) is the tensile stress and ε is the tensile strain in the elastic deformation regime of the material. The elastic modulus E of the retractor blades may varied (changed) varying the elastic modulus of the material(s) used to form or make the retractor blades. For example, the average elastic modulus (Eₐᵥₑ) of a given retractor blade may be varied by manufacturing the retractor blades with layered structures that combine at least one material with a “high” elastic modulus with another material with “low” elastic modulus or changing the amount of an additive, e.g. carbon fiber, in a composite matrix such as a polymer matrix.

Referring to FIGS. 8A-8B, a perspective view of the retractor blade 610 is shown in FIG. 8A and a cross-sectional view of section 8A-8A is shown in FIG. 8B. The retractor blade 610 is made from a pair of outer layers 616 and an inner layer (also referred to as in inner core) 618. Each of the outer layers 616 and the inner layer 618 has a thickness (not labeled in the figure) and the total thickness of the retractor blade 610 is t1. The thicknesses of each outer layer 616 may be about equal, or in the alternative, the thicknesses of each outer layer 616 may not be equal. The pair of outer layers 616 are made from a first material having a first elastic modulus (E₁) and the inner layer 618 is made from a second material having a second elastic modulus (E₂). In embodiments, the first elastic modulus is greater than the second elastic modulus (E₁>E₂). In other embodiments the first elastic modulus is less than the second elastic modulus (E₁<E₂). Although FIG. 8B shows the retractor blade 610 made from three layers, i.e., the pair of outer layers 616 and the inner layer 618, it should be appreciated that retractor blades 610, 620, 630 may be made from a single layer, two layers, four layers, ten layers, fifty layers, a hundred layers, or any number of layers between a single layer and a hundred layers, so long as a desired stiffness from a proximate end to a distal end for a given retractor blade is obtained. For example, the retractor blade 630 with length L3 may be made from a single layer of the first material with the first elastic modulus and retractor blade 620 with length L2 may be made from two outer layers 616 of the first material with the first elastic modulus and one inner layer 618 of the second material with the second elastic modulus. The combination of the two outer layers 616 made from the first material with the first elastic modulus and one inner layer 618 made from the second material with the second elastic provides a retractor blade 620 that has an modulus (E₆₂₀) that is less an elastic modulus of the retractor blade 630 (E₆₃₀), i.e., E₆₂₀<E₆₃₀. However, the shorter length of the retractor blade 620 compared to the retractor blade 630 results in the retractor blade 620 having approximately the same stiffness as the retractor blade 630 and thus deflects the same amount (d1) as the retractor blade 630 when the same load (P1) is applied to the distal end 624, 634 of each retractor blade 620, 630, respectively. Also, the retractor blade 610 with length L1 may be made from two outer layers 616 of the first material with the first elastic modulus and one inner layer 618 of the second material with the second elastic modulus. However, the combined thickness of the two outer layers 616 in the retractor blade 610 is less than the combined thickness of the two outer layers 616 of the retractor blade 620, and the thickness of the one inner layer 618 of the retractor blade 610 is greater than the thickness of the one inner layer 618 of the retractor blade 620. Accordingly, the retractor blade 610 contains a greater amount of the second material with the second elastic modulus than the retractor blade 620 and the elastic modulus of the retractor blade 610 (E₆₁₀) is less than the elastic modulus of the retractor blade 620, i.e., E₆₁₀<E₆₂₀. However, the shorter length of the retractor blade 610 compared to the retractor blade 620 results in the retractor blade 610 having the approximately same stiffness as the retractor blade 620 and thus deflects the same amount (d1) as the retractor blade 620 when the same load (P1) is applied to the distal end 614, 624 of each retractor blade 610, 620, respectively. In this manner, the three retractor blades 610, 620, 630 having the lengths L1, L2, L3 deflect the about same amount (d1) when a constant load (P1) is applied to the distal end of each retractor blade 610, 620, 630.

Although, the retractor blades 610 and 620 are described as having layers made from different materials with different elastic moduli, in embodiments, the blades may be made from a single layer of different materials, e.g., the retractor blades 610 and 620 have the same width W and thickness t1, but the retractor blade 620 is made from a single layer of a first material with a first elastic modulus and the retractor blade 610 is made from a single layer of a second material with a second elastic modulus that is less than the first elastic modulus. For example, such single layers may be made from polyether ether ketone (PEEK) with different amounts of impregnated carbon fibers that provide different elastic moduli. Also, the impregnated carbon fibers may or may not be impregnated unidirectional carbon fibers.

### Example

The embodiments of the retractor blades with different lengths exhibiting the same amount of deflection as described herein will be further clarified by the following example.

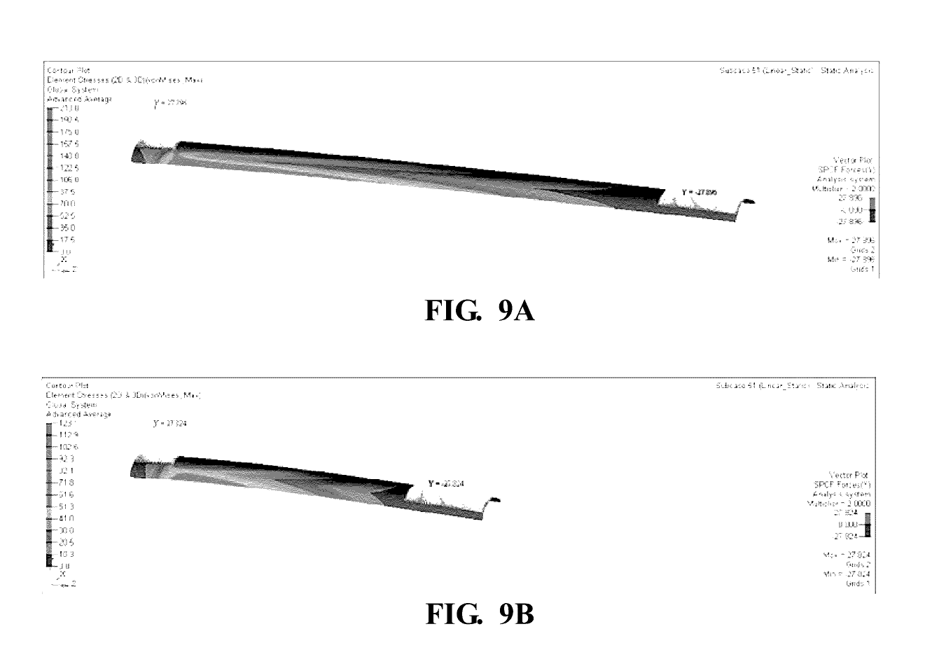

Computer modeling of two retractor blades having different lengths and with distal ends subjected to a constant load, or conversely, of two retractor blades having different lengths and with distal ends that exhibited a constant deflection, was performed. Particularly, retractor blades with lengths of 100 mm and 180 mm were modeled. Both retractor blades had the same width and an overall thickness of 2.75 mm. The 180 mm retractor blade was modeled as being made from a monolith (single layer) of carbon fiber impregnated PEEK (hereafter referred to as CF-PEEK) with an elastic modulus of about 20 gigapascals (GPa). A load or force of 27.9 newtons (N) applied to a distal end of the 180 retractor blade was required to deflect the distal end 4 mm (A=4 mm). A finite element (FE) model showing deflection as a function of resultant force applied to the 180 mm retractor blade is shown in FIG. 9A. The 100 mm retractor blade was modeled as being made from two outer layers of the CF-PEEK and inner layer (inner core) of PEEK with no carbon fiber impregnation (hereafter referred to simply as PEEK) with an elastic modulus of about 4 GPa. With each of the two outer layers of the CF-PEEK having a thickness of 0.5 mm and the inner layer of PEEK having a thickness of 1.75, a force of 98.47 N applied to the distal end of the 100 mm retractor blade was required to deflect the distal end 4 mm. With each of the two outer layers of the CF-PEEK having a thickness of 0.25 mm and the inner layer of PEEK having a thickness of 2.25 mm, a force of 57.6 N applied to the distal end of the 100 mm retractor blade was required to deflect the distal end 4 mm. With each of the two outer layers of the CF-PEEK having a thickness of 0.095 mm and the inner layer of PEEK having a thickness of 2.560 mm, a force of 27.9 N applied to the distal end of the 100 mm retractor blade was required to deflect the distal end 4 mm. Accordingly, the modeling provided two retractor blades with different lengths that had the same stiffness and exhibited the same amount of deflection (4 mm) when their distal ends were subjected to the same force (27.9 N). A FE model showing deflection as a function of resultant force applied to an optimized 100 mm retractor blade is shown in FIG. 9B and Table 1 summarizes the results of the modeling.

TABLE 1

| | | | | Distal End | Force on |
|---|---|---|---|---|---|
| | | | Thickness | Deflection | Distal |
| Sample # | Length (mm) | Layers | (mm) | (mm) | End (N) |
| 1 | 180 | Single layer: CF-PEEK | 2.75 | 4.0 | 27.9 |
| 2 | 100 | 1^{st} Outer layer: CF-PEEK | 0.50 | 4.0 | 98.5 |
| | | Inner Layer: PEEK | 1.75 | | |
| | | 2^{nd} Outer layer: CF-PEEK | 0.50 | | |
| 3 | 100 | 1^{st} Outer layer: CF-PEEK | 0.25 | 4.0 | 57.6 |
| | | Inner Layer: PEEK | 2.25 | | |
| | | 2^{nd} Outer layer: CF-PEEK | 0.25 | | |
| 4 | 100 | 1^{st} Outer layer: CF-PEEK | 0.095 | 4.0 | 27.9 |
| | | Inner Layer: PEEK | 2.560 | | |
| | | 2^{nd} Outer layer: CF-PEEK | 0.095 | | |

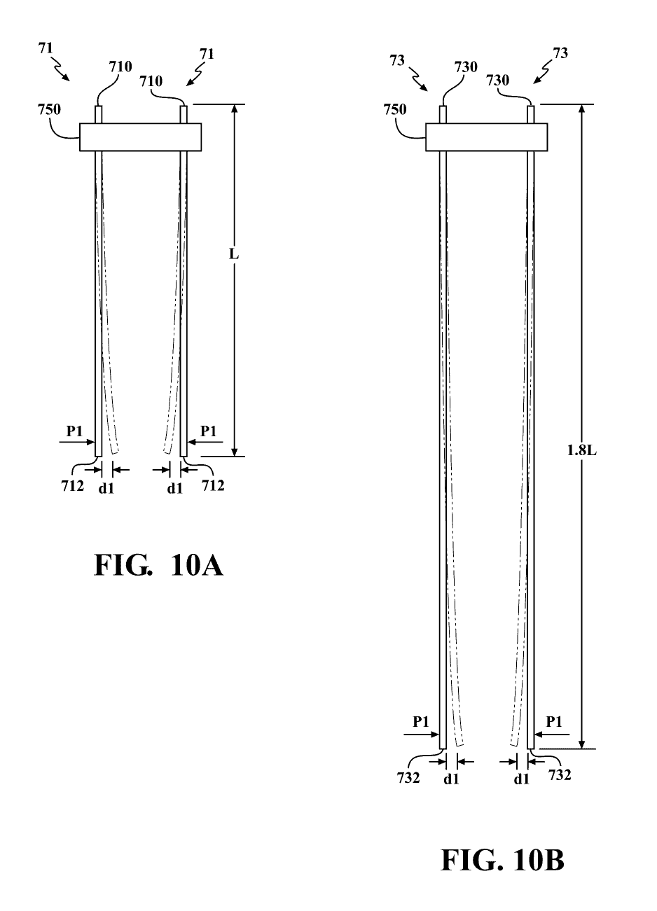

Referring to FIGS. 10A-10B, a retractor system or retractor blade kit with a frame 750 and two different sets of retractor blades 71, 73 according to the example described above is shown. In FIG. 10A, each of the retractor blades 71 have a length L and distal ends 710 deflect an amount d1 when a force P1 is applied thereto—proximal ends 712 are secured and held in fixed position relative to each other by the frame 750. The retractor blades 71 are made from a first material having a first elastic modulus and a second material having a second elastic modulus, the combination of which provides the retractor blade 71 with a stiffness such that the deflection d1 at the distal ends 712 is observed when the force P1 is applied thereto. In FIG. 10B, the retractor blades 73 have a length 1.8 L and distal ends 732 deflect the amount d1 when the force P1 is applied thereto—proximal ends 730 are secured and held in fixed position relative to each other by the frame 750. The retractor blades 73 are made only from the first material having the first elastic modulus, the first material with the first elastic modulus providing the retractor blade 73 with a stiffness that is about the same as the stiffness for retractor blade 71 and the deflection d1 at the distal ends 732 is observed when the force P1 is applied thereto.

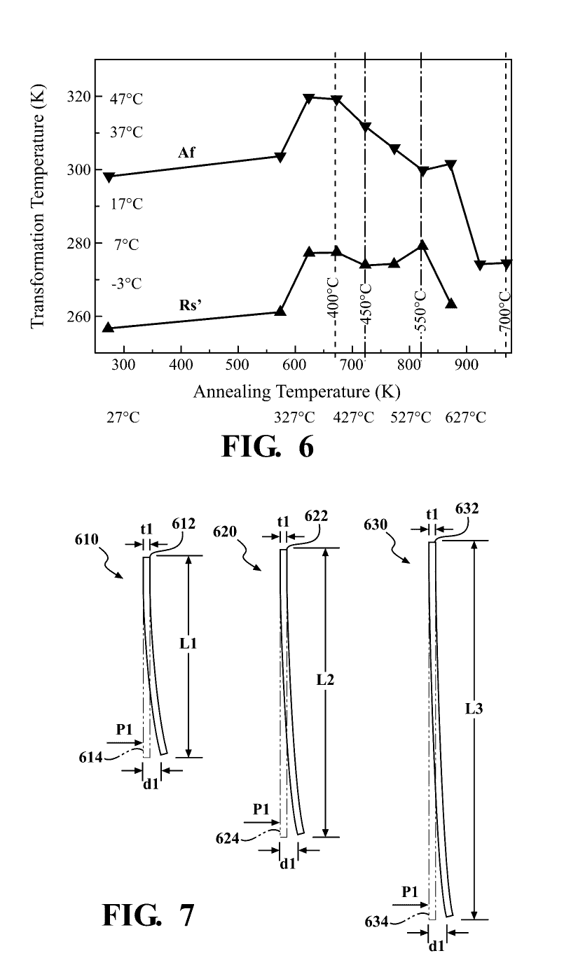

Based on the foregoing, it should now be understood that retractors, retractor blades, retractor systems and retractor blade kits described herein mitigate or lessen the effects of musculature load and/or reduce surgeon input, adjustment, manipulation, etc., of a retractor load during MIS procedures. In embodiments, retractor blades with an automatic toe-out mechanism pivot distal ends of retractor blades radially outward. Although the automatic toe-out mechanism described herein is made from a SMA, the automatic toe-out mechanism may be made from other materials illustratively including a shape memory polymer which may or may not be x-ray translucent. In other embodiments, retractor blades having different lengths exhibit the same amount of deflection when a constant load or force is applied to distal ends of the blades, thereby providing a surgeon with a retractor system or kit that provides constant retractor blade deflection independent of the length of retractor blades used during surgery. The retractor blades may or may not be made from materials that are x-ray translucent. It should be appreciated that retractor blades of different lengths and exhibiting the same stiffness may also have a toe-out mechanism as disclosed and described herein. That is, embodiments described in reference to FIGS. 1-6 may be combined with embodiments described in reference to FIGS. 7-10A.

Ranges can be expressed herein as from “about” one particular value, and/or to “about” another particular value. When such a range or value is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. Also, use of the term “approximately” refers to within +/−10%, preferably within /−5%, of a given value, endpoint, quantity, comparison, etc., however it will be understood the particular value, endpoint, quantity, comparison, etc., forms another embodiment.

It will be apparent to those skilled in the art that various modifications and variations can be made to the embodiments described herein without departing from the spirit and scope of the claimed subject matter. Thus it is intended that the specification cover the modifications and variations of the various embodiments described herein provided such modification and variations come within the scope of the appended claims and their equivalents.

1. A retractor blade comprising:
a blade having a proximal end, a distal end and a toe-out mechanism integrally formed with said blade and positioned between said proximal end and said distal end, wherein said toe-out mechanism pivots said distal end radially outward from an insert position to a toe-out position when activated.

2. The retractor blade of claim 1, wherein said toe-out mechanism is made from a shape-memory alloy (SMA) having a transformation temperature and pivots said distal end radially outward from said insert position to said toe-out position when a temperature of said SMA is raised from a first temperature to above said transformation temperature to a second temperature.

3. The retractor blade of claim 2, wherein said transformation temperature is between 0° C. and 40° C.

4. The retractor blade of claim 3, wherein said SMA is a Ni—Ti alloy and said transformation temperature is between 25° C. and 35° C.

5. The retractor blade of claim 2, wherein said blade comprises a first blade portion and a second blade portion, said first blade portion formed from said SMA and said second blade portion formed from a non-SMA material.

6. The retractor blade of claim 5, wherein said first blade portion extends generally parallel to said second blade portion along said blade.

7. The retractor blade of claim 5, wherein said first blade portion is co-axial with said second blade portion.

8. The retractor blade of claim 1, wherein said blade comprises a first blade portion, a second blade portion and a third blade portion, said first blade portion formed from said SMA and both of said second blade portion and third blade portion formed from a non-SMA material, wherein said first blade portion is positioned between said second blade portion and said third blade portion.

9. The retractor blade of claim 8, wherein said first blade portion is positioned from said distal end a distance between about 10 to 50 mm.

10. The retractor blade of claim 1, wherein said toe-out mechanism is activated when said toe-out mechanism is positioned within a surgical site of a patient and is heated from a first temperature that is less than or equal to about 26° C. to a second temperature is greater than or equal to about 35° C.

11. The retractor blade of claim 1, wherein said toe-out mechanism is activated when said toe-out mechanism is positioned outside a surgical site of a patient and is heated from a first temperature that is less than or equal to about 26° C. to a second temperature is greater than or equal to about 35° C.

12. The retractor blade of claim 1, further comprising a retractor frame, said proximal end of said blade attached to said retractor frame.

13. The retractor blade of claim 12, wherein said blade is a pair of blades with each of said pair of blades having said proximal end, said distal end and said toe-out mechanism between said proximal end and said distal end, said proximal ends of said pair of blades attached to said retractor frame.

14. A retractor blade system comprising:
a first blade having a first length, said first blade made from a first material having a first elastic modulus; and
a second blade having a second length that is less than said first length, said second blade made from said first material and a second material having a second elastic modulus, said second elastic modulus less than said first elastic modulus, said first blade and said second blade having approximately the same stiffness;
wherein said first blade and said second blade have approximately the same thickness and cross-sectional area, and a distal end of said first blade and a distal end of said second blade deflect an equal amount when an equal force is applied to said distal end of said first blade and said distal end of said second blade.

15. The retractor blade system of claim 14, wherein said first blade and said second blade have a layered structure, said second blade having a layer made from said first material and a layer made from said second material.

16. The retractor blade system of claim 15, wherein said first blade has a single layer of said first material.

17. The retractor blade system of claim 15, wherein said first blade has a single layer of said first material and said second blade has a pair of outer layers made from said first material and an inner layer made from said second material.

18. The retractor blade system of claim 14, wherein said first material is unidirectional carbon-fiber impregnated polyether ether ketone and said second material is polyether ether ketone.

19. The retractor blade system of claim 14, further comprising a third blade having a third length that is less than said second length, said third blade made from said first material and more of said second material than said second blade.

20. The retractor blade system of claim 19, wherein said second blade and said third blade each have a pair of outer layers made from said first material and an inner layer made from said second material, said pair of outer layers of said second blade being thicker than said pair of outer layers of said third blade.
